(19) United States
(12) Patent Application Publication
(10) Pub. No.: US 2005/0277146 A1
(43) Pub. Date: Dec. 15, 2005

(54) Method of amplifying template DNA molecule using strand-displacing DNA polymerase capable of carrying out isothermal amplification

(75) Inventors: Yasushi Shigemori, Kisarazu-shi (JP); Takehiko Shibata, Wako-shi (JP); Tsutomu Mikawa, Wako-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: AISIN SEIKI KABUSHIKI KAISHA, Kariya-shi (JP); RIKEN, Wako-shi (JP)

(21) Appl. No.: 11/138,443

(22) Filed: May 27, 2005

(30) Foreign Application Priority Data
May 28, 2004 (JP) 2004-159451

(51) Int. Cl. C12Q 1/68; C12P 19/34

(52) U.S. Cl. 435/6; 435/91.2

(57) ABSTRACT

A method of amplifying a template DNA molecule in an isothermal reaction that can reduce background noise is provided. A method of amplifying a template DNA molecule using a strand-displacing DNA polymerase capable of carrying out isothermal amplification includes a step of conducting an amplification reaction with the addition of a single-strand DNA binding protein (SSB) from an extreme thermophile.

### CROSS REFERENCE TO RELATED APPLICATIONS

This application is based on and claims priority under 35 U.S.C. §119 with respect to Japanese Patent Application 2004-159451, filed on May 28, 2004, the entire content of which is incorporated herein by reference.

### FIELD OF THE INVENTION

The present invention relates to a method of amplifying a template DNA molecule using a strand-displacing DNA polymerase capable of carrying out isothermal amplification.

### BACKGROUND ART

Heretofore, various methods for exponentially amplifying nucleic acids have been studied and developed in the art. Among them, in particular, the methods for effectively amplifying DNA molecules have been typically classified into those using a heat cycle with reaction-temperature variations and those carrying out their reactions under isothermal conditions.

An exemplary method using a heat cycle is a polymerase chain reaction (PCR) well known in the art (see, for example, Saiki et al., Science 230: 1350-1354, 1985). In the PCR, two primers having their respective sequences complementary to opposing strands of a target template DNA molecule are mixed with the template DNA molecule. The complementary strands of the template DNA molecule positioned between two primers being annealed on the template DNA molecule can be synthesized by performing typically 20 to 30 cycles of denaturing of the template DNA molecule, annealing of primers to the template DNA molecule, and extension of the primers with DNA polymerase (DNA replication).

In this method, a newly-synthesized strand can be used as an additional template DNA molecule, so that additional replication cycles with the same set of primers will allow the template DNA molecule to be amplified exponentially.

Also, in each cycle, there is a need for the use of a heat-stable DNA polymerase in order to withstand high processing heat required for the denaturation of the template DNA molecule. Furthermore, the DNA amplification with the PCR method should be carried out by subjecting a template DNA molecule (i.e., a nucleic acid sample) to a series of cycles because amplification reactions cannot proceed continuously.

On the other hand, as methods of carrying out amplification reactions of template DNA molecules under isothermal conditions, there have been known a strand displacement amplification (SDA) method (see, for example, Walker et al., Proc. Natl. Acad. Sci. USA 89: 392-396, 1992), a rolling circle amplification (RCA) method (see, for example, Lizardi et al., Nature Genetics 19: 225-232, 1998), and so on.

In the SDA method, the template DNA molecule is nicked by a restriction enzyme. Then, the DNA is amplified using the action of a DNA polymerase (strand-displacing DNA polymerase) that substitutes the nicked DNA fragments consecutively. On the other hand, in the RCA method, hybridization is carried out at the tip of an elongated strand that is synthesized from a primer annealed on the template DNA molecule, wherein a strand-displacing DNA polymerase displaces a preceding strand to undergo the hybridization. Therefore, in these methods, the amplification of a target DNA sequence is carried out continuously under isothermal conditions and thus there is no need of a heat cycle.

Such strand displacement enables continuous, linear or exponential amplification of a template DNA molecule under isothermal conditions.

Therefore, for example, compared with a method using a heat cycle, the RCA method has the following advantages: since the process for amplifying the template DNA molecule is simplified, the production amount of an amplification product can be efficiently increased; the length of the template DNA molecule which can be effectively amplified is not limited; there is no need of equipment for heat cycle; and so on.

Here, in the amplification reaction of a template DNA molecule, it is known that a single-strand DNA binding protein (hereinafter, referred to as SSB) is responsible for the efficiency etc. of the amplification reaction of the template DNA molecule.

The SSB has high sequence-nonspecific affinity to a single-stranded DNA (ssDNA). Usually, the SSB is required for the replication or recombination of DNA and the restoration of biological genomes. The SSB specifically stimulates its homologous DNA polymerase to increase the fidelity of DNA synthesis. Thus, the helical structure of the DNA becomes unstable, so that the ability of a DNA polymerase to move forward can be improved and the binding of the DNA polymerase can be also facilitated to organize and stabilize the origin of replication. In other words, it is known that the SSB acts as a replication-assisting protein (see, for example, JP No. H10-234389 Official Gazette, particularly descriptions in columns Nos. 0007 and 0017 thereof).

Various SSBs have been isolated from a wide variety of sources, ranging from bacteriophages to eukaryotes. For instance, JP No. H10-234389 Official Gazette discloses replication protein A-1 (rpa-1) derived from Saccharomyces cerevisiae, replication protein (rim-1) derived from a mitochondrial protein, gene-2.5 protein (gp2.5) derived from T7, protein p5 (p5) derived from bacteriophage φ29, gene-32 protein (gp32) derived from T4, and SSB of E. coli. In this document, furthermore, there is a description that SSB is added to an isothermal amplification reaction system for improving the efficiency of amplifying a template DNA molecule.

Furthermore, in US2004-170968A, SSB of E. coli is used as a strand-displacing factor useful for strand displacement replication of a template DNA molecule. In other words, in the presence of the strand-displacing factor, the RCA amplification of the template DNA molecule is performed using a strand-displacing DNA polymerase (e.g., DNA polymerase from bacteriophage φ29, etc.) which is capable of carrying out strand displacement replication.

These methods of amplifying a template DNA molecule using the strand-displacing DNA polymerase depend on the strand-displacing ability of the strand-displacing DNA polymerase that performs denaturation of the template DNA molecule. In addition, as the strand displacement can be facilitated by the replication-assisting protein and the strand-displacing factor, DNA fragments specific to the template DNA molecule can be efficiently amplified.

The methods disclosed in JPH10-234389A and WO 00/15849, in which an isothermal amplification reaction is carried out by the addition of SSB of E. coli, yeast, or the like have problems in that DNA fragments non-specific to the template DNA molecules tend to be amplified in addition to the efficient amplification of DNA fragments specific to the template DNA molecule.

A reason for such problems may be that, because the temperature of isothermal amplification is usually about 30 to about 60° C., a primer dimer tends to be formed easily, and as a result of the primer dimer formation, DNA fragments non-specific to the template DNA molecule tend to be amplified easily. The DNA fragments non-specific to the template DNA molecule may be a factor for lowering the accuracy of amplification products and become background noise which will be obstacles for subsequent experiments.

Thus, although the method for isothermal amplification of a template DNA molecule has been expected as a technology with high versatility because there is no need of a thermal cycle as in the case of PCR, etc., the method has limited usefulness due to the generation of background noise as described above.

Therefore, an object of the present invention is to provide a method of amplifying a template DNA molecule in an isothermal reaction, which is capable of preventing the generation of background noise.

### SUMMARY OF THE INVENTION

According to the present invention for attaining the above object, a method of amplifying a template DNA molecule is one that amplifies the template DNA molecule using a strand-displacing DNA polymerase capable of carrying out isothermal amplification. A first aspect of the inventive method is to carry out an amplification reaction with addition of a single-stranded DNA binding protein (SSB) of an extreme thermophile.

Since random primers are used in the method for isothermal amplification of DNA molecules such as the RCA method, it was difficult to reduce the generation of background noise by preventing the formation of a primer dimer.

Thus, the present inventors have studied intensively and found that, when an SSB of an extreme thermophile in particular among numerous SSBs was added to an isothermal amplification reaction system, amplification products specific to a template DNA were obtained as shown in Examples 1 and 2 to be described below, and that amplification products having high accuracy with little background noise were obtained (lane 6 in FIG. 1, lane 5 in FIG. 2, etc.).

Here, even though the SSB of the extreme thermophile is known in the art, the addition of such SSB to the isothermal amplification reaction system as in the invention has not been performed in the art. Furthermore, the finding that addition of the SSB to the isothermal amplification reaction system exerts an excellent effect to prevent the amplification of DNA fragments non-specific to the template DNA molecule was obtained by the present inventors for the first time.

On this account, an amplification method for a template DNA molecule in accordance with the above first aspect of the present invention is a method of amplifying the template DNA molecule, which is highly versatile and is not limited in application.

In a second aspect of the method of amplifying a template DNA molecule in accordance with the present invention, the strand-displacing DNA polymerase is +29-DNA polymerase.

According to the second aspect of the invention, an amplification product by preventing background noise favorably can be obtained as a result of easily carrying out an isothermal amplification reaction using a strand-displacing DNA polymerase which can be readily available and can be also easily handled.

In a third aspect of the method of amplifying a template DNA molecule in accordance with the present invention, the extreme thermophile is Thermus thermophilus HB8.

According the third aspect of the invention, there is no need of any specific facility or the like while an isothermal amplification reaction can be easily carried out because the extreme thermophile can be readily available and can be also easily handled. Therefore, an amplification product by preventing background noise favorably can be obtained.

In a fourth aspect of the method of amplifying a template DNA molecule in accordance with the present invention, a single-strand binding protein (SSB) of Thermus thermophilus SSB has a protein concentration in the range between 0.1 and 0.4 μg/μL.

In Examples 5 and 6 described later, a preferable amount of the SSB derived from Thermus thermophilus added to the isothermal amplification reaction was investigated.

As a result, according to the fourth aspect of the invention, it was confirmed that DNA fragments specific to the template DNA molecule could be efficiently obtained as far as the SSB of Thermus thermophilus SSB has a protein concentration in the range between 0.1 and 0.4 μg/μL.

Furthermore, the more the SSB is added, the more DNA fragments specific to the template DNA molecule can be obtained (see, Example 4 described later). However, considering that an excess amount of the SSB may be a factor causing lowering of the reaction efficiency, disturbing subsequent experiments, cost problems, and so on, it is preferable to set the upper limit of the amount of the added SSB to be approximately 0.4 μg/μL.

### BRIEF DESCRIPTION OF THE DRAWINGS

FIG. 1 is a diagram showing confirmation of the status of amplification of a DNA fragment of interest and the status of generation of background noise after carrying out an isothermal amplification for 24 hours by adding one kind of various proteins known as strand-displacing factor;

FIG. 2 is a diagram showing confirmation of the status of amplification of a DNA fragment of interest and the status of generation of background noise after carrying out an isothermal amplification for 18 hours by adding one kind of various proteins known as strand-displacing factor;

FIG. 3 is a diagram showing the results of electrophoresis of samples used in Example 2 that were treated with a restriction enzyme;

FIGS. 4a and 4b are diagrams showing the effect of the amount of added T. th. SSB on an isothermal amplification reaction;

FIGS. 5a and 5b are diagrams showing the results of studying a preferable amount of added T. th. SSB;

FIGS. 6a and 6b are diagram and graph showing confirmation of the specificity of an amplified DNA fragment obtained in Example 5 by using Southern Hybridization (FIG. 6a: the results of Southern hybridization, FIG. 6b: the results of the measurement of signal intensity); and

FIGS. 7a and 7b are diagrams showing the results of a detailed study of the reaction time in an isothermal amplification reaction.

### DETAILED DESCRIPTION

Hereinafter, the present invention will be described in detail.

The method of amplifying a template DNA molecule of the present invention is a method that amplifies the template DNA molecule using a strand-displacing DNA polymerase capable of carrying out isothermal amplification, and is characterized by carrying out the amplification reaction by the addition of a single-strand DNA binding protein (SSB) derived from an extreme thermophile.

The method of amplifying a template DNA molecule using a strand-displacing DNA polymerase depends on the strand-displacing ability of such DNA polymerase that denatures the template DNA molecule. Besides, the strand displacement can be facilitated by a strand-displacing factor.

As a preferable amplification method which corresponds to such a method, a rolling-circle amplification (RCA) method can be exemplified. In the following, the RCA method will be described as a method for isothermal amplification of a template DNA molecule.

For example, under isothermal conditions, from a plurality of random primers that are used as origins of replication and are annealed on a circular DNA molecule that is a template DNA molecule, a strand complementary to the circular DNA is replicated by a strand-displacing DNA polymerase. As the extension of a synthesized strand progresses, even if the synthesized strand reaches the replication origin of another random primer, the extension of the strand continues while removing another synthesized strand off by the strand-displacing activity of the strand-displacing DNA polymerase (branching). At this time, the synthesized strand being pealed off has an exposed portion on which the random primer can be annealed. That is, not only the circular DNA, but also the synthesized strand being pealed off can be provided as a template DNA molecule to form an additional synthesized DNA strand, resulting in exponential amplification.

In this case, a random hexamer or the like can be suitably used as a random primer. Other examples of the primer include those which can be specifically annealed on their respective portions of the template DNA molecule at preset temperatures. The primer may be used independently or in combination with the random primer described above.

The primer may be designed such that a desired region can be amplified on the basis of a target nucleic acid sequence, and may be designed by, for example, a primer-design support software or the like. In the case of the random primer, it is designed to have a random sequence.

The primer thus designed may be chemically synthesized. For example, a primer may be chemically synthesized in a solid phase synthesis using a phosphoramidite method which is known in the art. It may be also possible to automatically synthesize a primer having a desired nucleic acid sequence by a commercially available automatic nucleic acid synthesizer. The primer after the synthesis may be purified by any of methods known in the art, such as HPLC, if required.

Here, the term “isothermal” as used in the isothermal amplification of the present invention refers to carrying out the amplification reaction by controlling the reaction temperature at a constant temperature, unlike the PCR method where the reaction temperature is varied at each step of DNA denaturation, annealing, and strand extension. The constant temperature for the amplification reaction is preferably less than 60° C., more preferably less than 45° C., and further preferably less than 37° C. This temperature can be appropriately determined depending on the strand-displacing DNA polymerase to be applied. For example, when φ29-DNA polymerase derived from bacteriophage is used, the amplification reaction can be preferably carried out at temperatures ranging from 25 to 42° C., preferably from 30 to 37° C., and more preferably from 30 to 34° C.

In a thermostated chamber such as an incubator set to be kept at a constant temperature, a sample is incubated for 4 to 24 hours, preferably for 6 to 24 hours, and more preferably for approximately 15 to approximately 24 hours to carry out amplification reaction of a template DNA molecule.

As the strand-displacing DNA polymerase of the invention, a preferable one is exemplified by +29-DNA polymerase derived from bacteriophage (Blanco et al., U.S. Pat. Nos. 5,198,543 and 5,001,050) but is not limited thereto. Examples of the strand-displacing DNA polymerase include DNA polymerase of the Bst large fragment (Exo(−)Bst (Aliotta et al., Genet. Anal. (Holland) 12: 185-195 (1996) and Exo(−)BcaDNA polymerase (Walker and Linn, Clinical Chemistry 42: 1604-1608 (1996)), phage M2 DNA polymerase (Matsumoto et al., Gene 84: 247 (1989)), phage φPRD1 DNA polymerase (Jung et al., Proc. Natl. Acad. Sci. USA 84: 8287 (1987)), VENT® DNA polymerase (Kong et al., J. Biol. Chem. 268: 1965-1975 (1993)), Klenow fragment of DNA polymerase I (Jacobsen et al., Eur. J. Biochem. 45: 623-627 (1974)), T5 DNA polymerase (Chatterjee et al., Gene 97: 13-19 (1991)), SEQUENASE® (manufactured by US Biochemicals Corp.), PRD1 DNA polymerase (Zhu and Ito, Biochem. Biophys. Acta. 1219: 267-276 (1994)), T4 DNA polymerase holoenzyme (Kaboord and Benkovic, Curr. Biol. 5: 149-157 (1995)), etc.

The template DNA molecule of the invention may be preferably a circular DNA molecule, but is not limited thereto. A linear DNA molecule may be also used. In the case of the RCA amplification method, the circular DNA is preferable because of its amplification efficiency.

The template DNA molecule used may be of a single or a double strand. In addition, the template DNA molecule may be any of various DNA molecules including naturally-occurring DNA molecules such as plasmid DNA and genome DNA of eucaryotic and procaryotic organisms, and artificially-prepared DNA molecules such as bacterial artificial chromosomal (BAC) DNA, phagemid, cosmid, etc. Furthermore, any of synthetic DNAs such as oligonucleotides may be used as the template DNA molecule.

The extreme thermophile of the present invention is a bacterium which is capable of growing at high temperatures, with optimal growth temperatures of, for example, 45° C. to 80° C. Preferable extreme thermophiles can be exemplified by Thermus thermophilus HB8, Thermus aquaticus, etc., but not limited thereto.

As the single-strand DNA binding protein (SSB), a protein extracted from an extreme thermophile is used. Preferably, the protein is extracted from one of the two extreme thermophile species described above. An SSB other than the extreme thermophile species, for example, the SSB derived from E. coli is not suitable because of the following reason: when such SSB is used, in amplification products obtained after the isothermal amplification reaction, background noise that is non-specific to the template DNA molecule is observed.

The SSB of these extreme thermophile species can be easily purified by using a known host-expression vector system of E. coli or the like. For example, the host E. coli is transformed by an expression vector in which a gene that encodes such SSB is introduced by a known method, and is then incubated to express the SSB. Subsequently, the host E. coli is homogenized and then treated with heat. Under these conditions, proteins derived from E. coli other than the SSB are denatured and undergo thermal aggregation, so that they can be isolated and removed by centrifugation or the like. Therefore, the SSB which is not denatured under heat can be isolated as a soluble fraction from the E. coli proteins and then purified using affinity chromatography or the like.

At this time, as the SSB is derived from the extreme thermophile, it has a stable structure at room temperature and also has high stability against an organic solvent. Thus, the above purification step may be carried out at room temperature.

In addition, the host cells are not limited to E. coli only. Eukaryotic cells such as those of Saccharomyces cerevisiae and insects (Sf9 cells) may be used.

Furthermore, the expression vector may be any of vectors as far as it contains a sequence of a multiple cloning site or the like having at least one restriction enzyme site where a gene that encodes the promoter sequence and the SSB of the extreme thermophile can be inserted, and can be expressed in the above host cell. As a suitable promoter, for example, T71ac promoter is used preferably.

Furthermore, the expression vector may contain any of other base sequences known in the art. The other base sequences known in the art include, but not specifically limited to, a stabilizing leader sequence that imparts the stability of an expression product, a signal sequence that imparts the secretion of the expression product, and marker sequences that provide transformed host cells with phenotypic selection, such as the sequences of neomycin resistance gene, kanamycin resistance gene, chloramphenicol resistance gene, ampicillin resistance gene, hygromycin resistance gene, and the like.

The expression vector may be a commercially available E. coli expression vector (e.g., pET Protein Expression System, manufactured by Novagen, Inc.). Furthermore, expression vectors which appropriately incorporate desired sequences may be prepared and used.

The concentration of the extreme-thermophile SSB added to the isothermal amplification system is not specifically limited. However, when the concentration is in a range of approximately 0.1 to approximately 0.4 μg/μL, DNA fragments specific to a template DNA molecule can be obtained efficiently. Preferably, by using the concentration in a range of approximately 0.3 to 0.4 μg/μL, the DNA fragments specific to the template DNA molecule can be efficiently obtained in a state where generation of background noise such as DNA fragments non-specific to the template DNA molecule is prevented.

### EXAMPLES

### Example 1

Hereinafter, a method of amplifying a template DNA molecule of the present invention will be described with reference to the drawings. An RCA method will be explained as the method of amplification.

Using the isothermal amplification reaction system of Templiphi DNA Amplification Kit (manufactured by Amersham Biosceiences), status of amplification of DNA fragments derived from a target DNA and status of background noise generation were confirmed for Samples 1-1 to 1-14 below by the addition of any one of a variety of proteins associated with recombination (Rec0, RecA, SSB, and T4 gene 32) known as a strand-displacing factor or a replication-assisting protein.

Sample 1-1 was Control 1-1 that was used as a positive control. Using 1 ng of pUC19 DNA as a template DNA molecule, φ29 DNA polymerase as a strand-displacing DNA polymerase, and a random hexamer as a random primer, isothermal amplification of the template DNA molecule was conducted for a reaction time set for 24 hours according to the manufacturer's instruction.
For Samples 1-2 to 1-7, amplification reaction was performed by adding the following substances to the reaction system of Sample 1-1 to make the total volume of each reaction solution to 10 μL:

Sample 1-2: 3.0 μg of Rec0 derived from an extreme thermophile Thermus thermophilus HB8 (hereinafter, referred to as T. th.),
Sample 1-3: 3.0 μg of RecA derived from E. coli,
Sample 1-4: 3.0 μg of T. th. RecA,
Sample 1-5: 3.0 μg of E. coli SSB,
Sample 1-6: 3.0 μg of T. th. SSB, and
Sample 1-7: 3.0 μg of T4 gene 32.

Amplification reaction for the Samples 1-8 to 1-14 was conducted under the same conditions as the Samples 1-1 to 1-7 except for the absence of pUC19 DNA as the template DNA molecule.

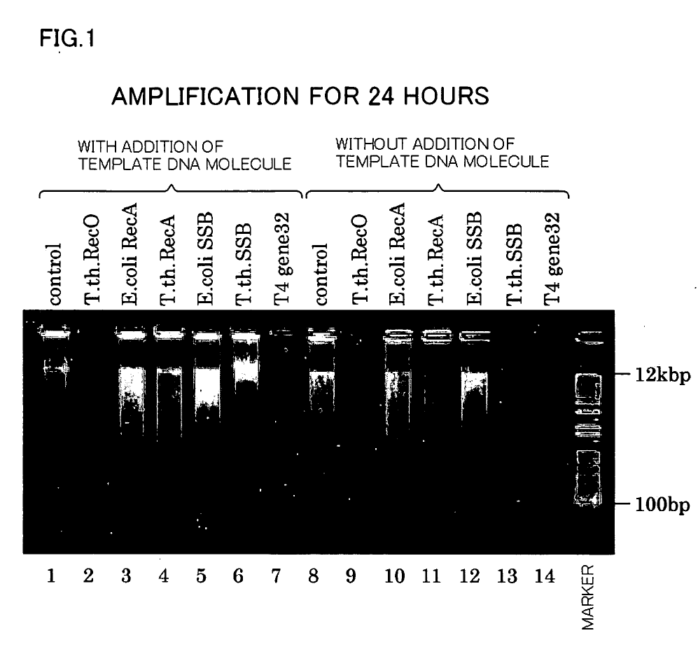

After the amplification reaction, the RCA reaction was terminated by heat denaturation at 65° C. for 10 minutes, and 5 μL of each reaction solution was subjected to 1% agarose electrophoresis. Electrophoresis was conducted at 4.5 V/cm for 45 minutes according to a standard method, and the results of ethidium bromide staining performed after electrophoresis are shown in FIG. 1. In FIG. 1, lanes 1 to 14 each correspond to the reaction solutions of Samples 1-1 to 1-14 subjected to the electrophoresis.

From the results thus obtained, it was found that only in Control 1-1 (lane 1) and Sample 1-6 (lane 6) that was subjected to the amplification reaction with the addition of T. th. SSB, amplification of DNA fragments specific to pUC19 DNA as the template DNA molecule was observed.

Amplification products observed in the amplification reaction without the addition of the template DNA molecule as in Samples 1-8 (lane 8) and 1-10 to 1-12 (lanes 10 to 12) are background noise having no relation to the template DNA molecule. In Samples 1-3 to 1-5 (lanes 3 to 5) to which the recombination-associated proteins other than SSB of the extreme thermophile were added, amplification products having the same size as those of amplification products obtained by the amplification reaction without the addition of, the template DNA molecule as in Samples 1-8 (lane 8) and 1-10 to 1-12 (lanes 10 to 12) were observed. These amplification products are likely to be background noise caused by, for example, the formation of a primer dimer and, therefore, are not the DNA fragments specific to the template DNA molecule.

Therefore, it is considered that the inhibition of template DNA molecule amplification takes place in the samples subjected to amplification reaction with the addition of the recombination-associated proteins other than the SSB of the extreme thermophile, whereas such inhibition of amplification can be reduced in the samples subjected to amplification reaction with the addition of T. th. SSB.

### Example 2

Using the same reaction system as in Example 1, the isothermal amplification of a template DNA molecule for Samples 2-1 to 2-14 below was conducted for a reaction time set for 18 hours:

Sample 2-1 was Control 2-1 (same as Control 1-1).

For Samples 2-2 to 2-7, amplification reaction was performed by adding the following substances to the reaction system of Sample 2-1 to make the total volume of each reaction solution to 10 μL:
Sample 2-2: 3.0 μg of T. th. Rec0,
Sample 2-3: 3.0 μg of T. th. RecA,
Sample 2-4: 3.0 μg of E. coli RecA,
Sample 2-5: 3.0 μg of T. th. SSB,
Sample 2-6: 3.0 μg of E. coli SSB, and
Sample 2-7: 3.0 μg of T4 gene 32.

Amplification reaction for Samples 2-8 to 2-14 was conducted under the same conditions as the Samples 2-1 to 2-7 except for the absence of the template DNA molecule.

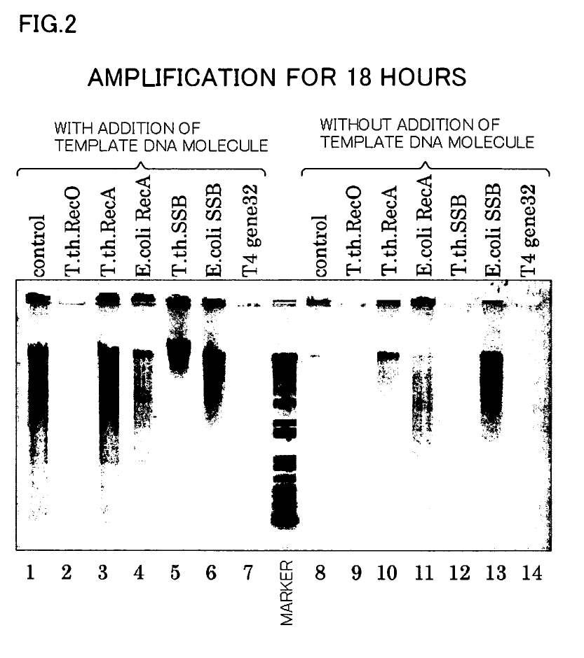

After amplification reaction, 1% agarose electrophoresis was conducted in the same way as in Example 1 and the results are shown in FIG. 2. In FIG. 2, lanes 1 to 14 each correspond to the reaction solutions of Samples 2-1 to 2-14 subjected to electrophoresis.

From the results thus obtained, similar to the results of Example 1, it was found that only in Control 2-1 (lane 1) and Sample 2-5 (lane 5) that was subjected to amplification reaction with the addition of T. th. SSB, amplification of DNA fragments specific to pUC19 DNA as the template DNA molecule was observed.

In Samples 2-3,2-4, and 2-6 (lanes 3, 4, and 6), amplification products having the same size as those of the amplification products obtained by the amplification reaction without addition of the template DNA molecule as in Samples 2-10, 2-11, and 2-13 (lanes 10, 11, and 13) were observed. These amplification products are likely to be background noise caused by a primer dimer and so on and, therefore, are not the DNA fragments specific to the template DNA molecule.

Since the same results were obtained from Examples 1 and 2 even though the reaction time was changed, it is considered that no change in the action of T. th. SSB is observed by the change in the reaction time.

### Example 3

For Samples 2-1 to 2-14 used in Example 2, obtained after isothermal amplification, 5 μL of each amplification reaction solution was subjected to a restriction enzyme (EcoRI) treatment (Samples 3-1 to 3-14). The restriction enzyme treatment was performed by using 10 units of the restriction enzyme at 37° C. for a reaction time of 2 hours.

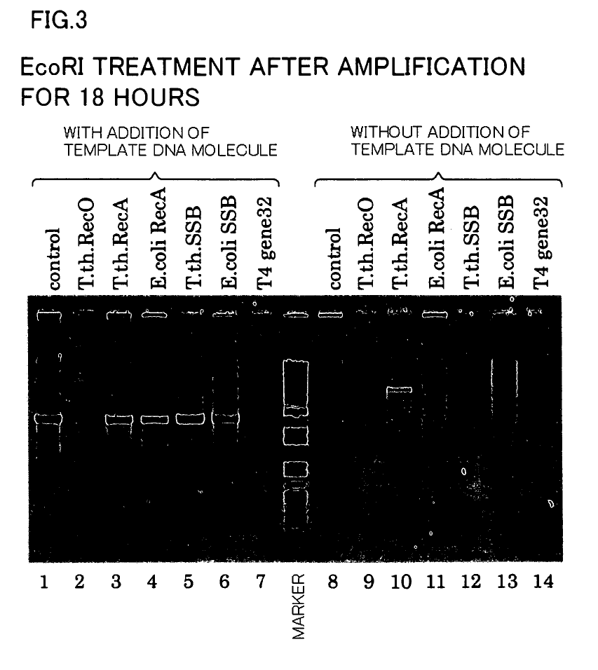

Following the restriction enzyme treatment, 5 μL of each restriction enzyme-treated solution was subjected to 1% agarose electrophoresis. Electrophoresis was conducted in the same way as in Example 1. The results are shown in FIG. 3. In FIG. 3, lanes 1 to 14 each correspond to the reaction solutions of Samples 3-1 to 3-14 subjected to the electrophoresis.

From these results, it was found that DNA fragments specific to pUC19 DNA as the template DNA molecule were contained in Samples 3-1 and 3-3 to 3-6 (see the lanes 1 and 3 to 6).

However, it was confirmed, from the results of Sample 3-10 (T. th. RecA) subjected to amplification reaction without the addition of the template DNA molecule, that DNA fragments non-specific to the template DNA molecule were contained in Sample 3-3 (T. th. RecA) by the amplification reaction.

Moreover, DNA molecules that were not cleaved by the restriction enzyme EcoRI were observed in the well of agarose gel of the lane 3 (Sample 3-3) in FIG. 3. These DNA molecules are considered to be DNA fragments produced as a result of the amplification that is non-specific to the template DNA molecule. The same holds true for Samples 3-4 (E. coli RecA) and 3-6 (E. coli SSB).

From the above results, it has been shown that only the samples to which T. th. SSB was added can prevent the amplification of DNA fragments that are non-specific to pUC19 DNA as the template DNA molecule (see Samples 3-5 and 3-12).

### Example 4

The effect of the amount of added T. th. SSB on the amount of an amplification product was examined in an isothermal amplification reaction system (Samples 4-1 to 4-7).

Sample 4-1 was prepared in the same way as Sample 1-1 used in Example 1.

Samples 4-2 to 4-6 were prepared by adding the following amount of T. th. SSB to the reaction system of Sample 1-1: 3.0 μg (0.3 μg/μL) for Sample 4-2, 1.5 μg (0.15 μg/μL) for Sample 4-3, 0.8 μg (0.08 μg/μL) for Sample 4-4, 0.4 μg (0.04 μg/μL) for Sample 4-5, and 0.2 μg (0.02 μg/μL) for Sample 4-6. Sample 4-7 was prepared by adding only a T. th. SSB lysate (50 mM Tris-HCl (pH 7.5), 1.5 M KCl, 1.0 mM EDTA, 0.5 mM DTT, and 50% glycerol; without T. th. SSB) to the reaction system of Sample 1-1. The total amount of each reaction solution was adjusted to 10 μL to conduct amplification reaction. The amplification reaction was conducted according to Example 1.

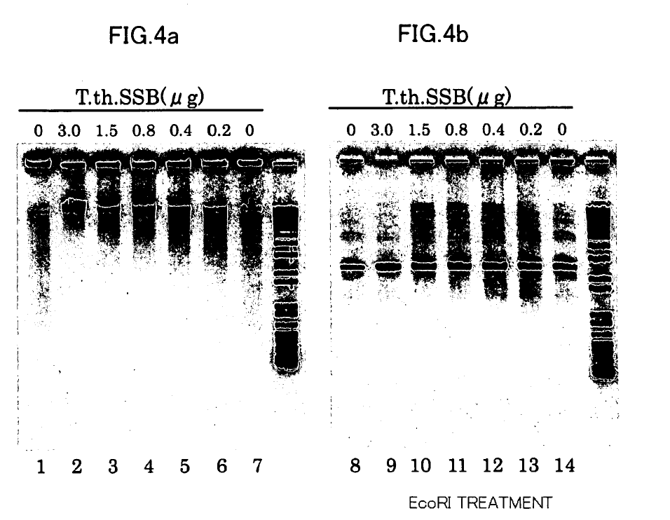

After the amplification reaction, 5 μL of each reaction solution was subjected to 1% agarose electrophoresis. Electrophoresis was conducted in the same way as in Example 1. The results are shown in FIG. 4a. In FIG. 4a, lanes 1 to 7 each correspond to the reaction solutions of Samples 4-1 to 4-7 subjected to the electrophoresis.

Samples 4-1 to 4-7 after the isothermal amplification were subjected to a restriction enzyme (EcoRI) treatment (Samples 4-8 to 4-14). The composition of the reaction solution was the same as that shown in the restriction enzyme treatment of Example 3.

Following the restriction enzyme treatment, 5 μL of each restriction enzyme-treated solution was subjected to 1% agarose electrophoresis. Electrophoresis was conducted in the same way as in Example 1. The results are shown in FIG. 4b. In FIG. 4b, lanes 8 to 14 each correspond to the reaction solutions of Samples 4-8 to 4-14 subjected to the electrophoresis.

From the above results, it was shown that, when 0.02 to 0.3 μg/μL of T. th. SSB was added to the amplification reaction system, the amount of DNA fragments specific to pUC19 DNA as the template DNA molecule increased, and that the amplification efficiency was improved with the amount of added T. th. SSB.

### Example 5

The desirable amount of T. th. SSB added was examined in an isothermal amplification system (Samples 5-1 to 5-16).

Sample 5-1 was prepared in the same way as in Sample 1-1 used in Example 1.

Samples 5-1 to 5-5 were prepared by adding the following amount of T. th. SSB to the reaction system of Sample 1-1: 1.0 μg (0.1 μg/μL) for Sample 5-2, 2.0 μg (0.2 μg/μL) for Sample 5-3, 3.0 μg (0.3 μg/μL) for Sample 5-4, and 4.0 μg (0.4 μg/μL) for Sample 5-5. Sample 5-6 was prepared by adding only a T. th. SSB lysate (50 mM Tris-HCl (pH 7.5), 1.5 M KCl, 1.0 mM EDTA, and 0.5 mM DTT, 50% glycerol; without T. th. SSB) to the reaction system of Sample 1-1. The total amount of each reaction solution was adjusted to 10 μL to conduct amplification reaction.

Samples 5-7 to 5-12 were subjected to amplification reaction under the same conditions as those of Samples 5-1 to 5-6 except for the absence of the template DNA molecule.

The amplification reaction was conducted according to Example 1 except that the amplification time was set for 18 hours.

Besides, Samples 5-13 to 5-16 were subjected to the amplification reaction under the same conditions as those of Samples 5-1, 5-4, 5-7 and 5-10, respectively, except that the amplification time was set for 14 hours.

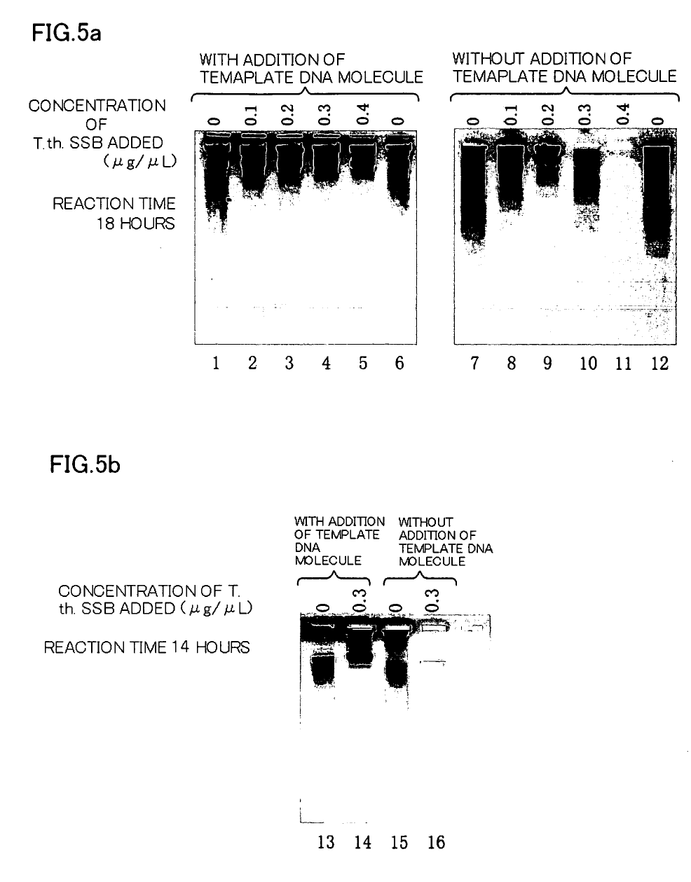

After the amplification reaction, 8 μL of each reaction solution was subjected to 1.2% agarose electrophoresis. The electrophoresis was conducted in the same way as in Example 1. The results are shown in FIGS. 5a and 5b. In FIGS. 5a and 5b, lanes 1 to 16 each correspond to the reaction solutions of Samples 5-1 to 5-16 subjected to the electrophoresis.

From the above results, it was found that, by the addition of 0.1 to 0.4 μg/μL of T. th. SSB, efficient amplification of DNA fragments specific to pUC19 DNA as the template DNA molecule was obtained in Samples 5-2 to 5-5 (see the lanes 2 to 5 in FIG. 5a).

Here, as described in Example 3, DNA molecules are sometimes observed in the well of agarose gel as a result of electrophoresis. However, these DNA molecules are considered to be DNA fragments produced as a result of amplification that is non-specific to the template DNA molecule.

As described above, in the present invention, the addition of T. th. SSB was found to enable prevention of the amplification of DNA fragments non-specific to pUC19 DNA as the template DNA molecule. However, in Samples 5-10 to 5-11 (see the lanes 10 to 11 in FIG. 5a) in which 0.3 to 0.4 μg/μL of T. th. SSB was added but no template DNA molecule was added, almost no DNA molecule was observed in the well of agarose gel. Therefore, it was found that, by the addition of T. th. SSB, preferably in the amount of 0.3 to 0.4 μg/μL, DNA fragments specific to pUC19 DNA as the template DNA molecule were obtained with further reduced background noise.

Moreover, from the results shown in FIG. 5b, it is considered that no change in the action of T. th. SSB is observed by the change in the reaction time, because almost the same results are obtained in Samples 5-1, 5-4, 5-7, and 5-10 even though the amplification reaction time is changed.

### Example 6

The specificity of the amplified DNA fragments obtained in Example 5 was confirmed by Southern Hybridization.

To a nylon membrane (Biodyne B membrane: manufactured by Nihon Pall Ltd.), aliquots of, 1.5 μL of each amplification reaction solution of Samples 5-1 to 5-7 and 5-10 in Example 5 were spotted, which were in turn used as Samples 6-1 to 6-7, respectively.

As a probe, a 100 nanogram specimen of pUC19 DNA labeled with 32P using the Random Primer DNA Labeling Kit (manufactured by TAKARA SHUZO) was used. Hybridization reaction was performed by using 2× Prehybridization/Hybridization Solution (manufactured by GibcoBRL) and by bringing the above-described membrane spotted with the amplification reaction solution into contact with the labeled probe according to the manufacturer's instruction.

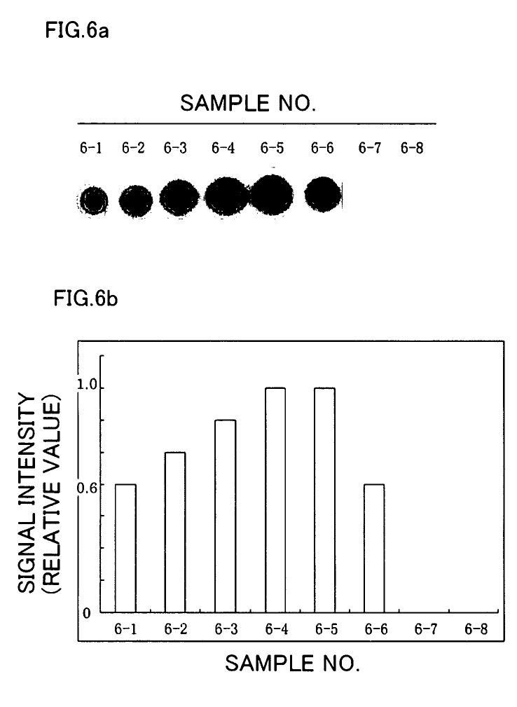

After the reaction, the reaction mixture was washed twice each with a washing buffer (0.1×SSC, 0.5% SDS) at 68° C. for 30 minutes. Next, an analysis was conducted by detecting signals using an image analyzer BAS2000 (manufactured by FUJIFILM Co., Ltd.) according to the manufacturer's instruction. The results of Southern Hybridization and the measurement of signal intensity are shown in FIG. 6a and FIG. 6b, respectively.

As shown in the results in FIG. 6a, no signal is detected from Samples 6-7 and 6-8 without the addition of the template DNA molecule. Also, from the signals of Samples 6-2 to 6-5 with the addition of both the template DNA molecule and T. th. SSB, it was observed that the amount of pUC19 DNA as the template DNA molecule was increased as compared with the signals of Samples 6-1 and 6-6 without the addition of T. th. SSB. Therefore, improvement in the amplification efficiency has been confirmed.

In particular, from the results of measured signal intensity shown in FIG. 6b, it was found that the signals of Samples 6-4 and 6-5 (T. th. SSB concentration of 0.3 to 0.4 μg/μL) had signal intensity approximately twice the signal intensity of the signals of Samples 6-1 and 6-6 without the addition of T. th. SSB. Therefore, it has been confirmed that the amplification efficiency can be improved by minimizing the background noise as long as T. th. SSB has the concentration within the range of 0.3 to 0.4 μg/μL. In this way, optimization of the concentration of added T. th. SSB was acomplished.

### Example 7

Using Sample 2-5 with the added T. th. SSB in Example 2, a further detailed study of the reaction time was conducted (Samples 7-1 to 7-3). The reaction times for isothermal amplification reaction were set for 15 hours for Sample 7-1, 17 hours for Sample 7-2, and 21 hours for Sample 7-3. Conditions other than the isothermal amplification reaction time were the same as in Example 1.

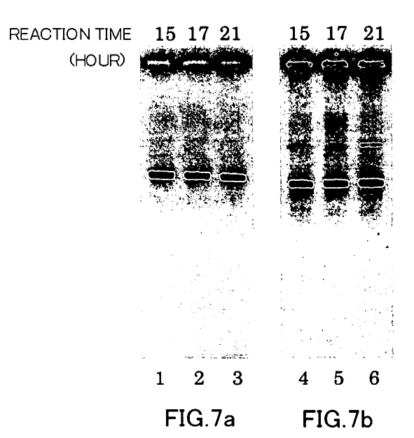

After the amplification reaction, 1% agarose electrophoresis was conducted in the same way as in Example 1 and the results are shown in FIG. 7a. In FIG. 7a, lanes 1 to 3 each correspond to the reaction solutions of Samples 7-1 to 7-3 subjected to the electrophoresis.

Using Sample 4-7 without the addition of T. th. SSB in Example 4, a further detailed study of the reaction time was conducted (Samples 7-4 to 7-6). The reaction times for isothermal amplification reaction were set for 15 hours for Sample 7-4, 17 hours for Sample 7-5, and 21 hours for Sample 7-6. Conditions other than the isothermal amplification reaction time were the same as in Example 1.

After the amplification reaction, 1% agarose electrophoresis was conducted in the same way as in Example 1 and the results are shown in FIG. 7b. In FIG. 7b, lanes 4 to 6 each correspond to the reaction solutions of Samples 7-4 to 7-6 subjected to the electrophoresis.

From the results thus obtained, it is considered that no change in the action of T. th. SSB is observed by the change in the reaction time because approximately the same degree of DNA fragments specific to the template DNA molecule is obtained even though the reaction time is changed.

Here, DNA molecules were observed in the well of agarose gel in FIG. 7b (a sample without the addition of T. th. SSB). These DNA molecules are considered to be DNA fragments produced as a result of amplification that is non-specific to the template DNA molecule.

On the other hand, such DNA molecules are hardly observed in the well of agarose gel in FIG. 7a. This is considered probably due to the following reason: by the addition of T. th. SSB to the isothermal amplification reaction system, strand displacement proceeds appropriately to make the inhibition action of strand extension of the DNA polymerase difficult, which facilitates the amplification of DNA fragments specific to the template DNA molecule.

The method of amplifying a template DNA molecule of the present invention is a method which enables the amplification of a DNA fragment specific to the template DNA molecule as well as reduction in the background noise. Therefore, the method is useful as a general method in molecular biology, for example, as a method useful for preparing DNA in a large amount from a small amount of a sample extracted from a trace amount of microorganisms collected from the environment in order to analyze genotype, or as a method of preparing DNA for DNA sequencing. Moreover, the method of the present invention can provide a highly versatile method of preparing DNA that can be applied to a variety of usages such as preparation of DNA for immobilizing a DNA chip from a small amount of a sample extracted from animal or plant cells.

### Alternative Embodiment

Although the embodiments described above illustrated examples with the addition of an extreme thermophile to an isothermal amplification system in an RCA method, the present invention is not limited to them and can utilize, for example, the addition of SSB of an extreme thermophile in a strand displacement amplification (SDA) method.

In this case, it is expected that the amount of DNA fragments specific to a template DNA molecule increases and amplification efficiency is improved with an increase in the amount of the SSB added, as shown in FIG. 4.

1. A method of amplifying a template DNA molecule using a strand-displacing DNA polymerase capable of carrying out isothermal amplification, wherein the amplification reaction is carried out by the addition of a single-strand DNA binding protein (SSB) obtained from an extreme thermophile.

2. The method of amplifying a template DNA molecule using a strand-displacing DNA polymerase capable of carrying out isothermal amplification according to claim 1, wherein the strand-displacing DNA polymerase is φ29DNA polymerase.

3. The method of amplifying a template DNA molecule using a strand-displacing DNA polymerase capable of carrying out isothermal amplification according to claim 1, wherein the extreme thermophile is Thermus thermophilus HB8.

4. The method of amplifying a template DNA molecule using a strand-displacing DNA polymerase capable of carrying out isothermal amplification according to claim 2, wherein the extreme thermophile is Thermus thermophilus HB8.

5. The method of amplifying a template DNA molecule using a strand-displacing DNA polymerase capable of carrying out isothermal amplification according to claim 3, wherein the SSB of Thermus thermophilus HB8 has a protein concentration in a range of 0.1 to 0.4 μg/μL.

6. The method of amplifying a template DNA molecule using a strand-displacing DNA polymerase capable of carrying out isothermal amplification according to claim 4, wherein the SSB of Thermus thermophilus HB8 has a protein concentration in a range of 0.1 to 0.4 μg/μL.

7. The method of amplifying a template DNA molecule using a strand-displacing DNA polymerase capable of carrying out isothermal amplification according to claim 1, wherein the amplification reaction is a rolling circle amplification (RCA) reaction.

8. The method of amplifying a template DNA molecule using a strand-displacing DNA polymerase capable of carrying out isothermal amplification according to claim 1, wherein the template DNA molecule is a circular DNA molecule.

9. A method of amplifying a template DNA molecule using a strand-displacing DNA polymerase capable of carrying out isothermal amplification, comprising the steps of:
annealing a primer to the template DNA molecule; and
extending a complementary strand of the template DNA at the annealed primer as a replication origin by the strand-displacing DNA polymerase, wherein the strand-displacing DNA polymerase is made to act on the template DNA molecule that was annealed with the primer in the above-mentioned annealing step, in which, when the extension reaction portion comes into contact with an already-synthesized complementary strand portion, the extension reaction proceeds while tearing the already-synthesized strand off by the strand-displacing activity,
wherein an SSB obtained from an extreme thermophile is added in the extension step.

10. The method of amplifying a template DNA molecule using a strand-displacing DNA polymerase capable of carrying out isothermal amplification according to claim 9, wherein the strand-displacing DNA polymerase is φ29DNA polymerase.

11. The method of amplifying a template DNA molecule using a strand-displacing DNA polymerase capable of carrying out isothermal amplification according to claim 9, wherein the extreme thermophile is Thermus thermophilus HB8.

12. The method of amplifying a template DNA molecule using a strand-displacing DNA polymerase capable of carrying out isothermal amplification according to claim 11, wherein the SSB of Thermus thermophilus HB8 has a protein concentration in a range of 0.1 to 0.4 μg/μL.

13. The method of amplifying a template DNA molecule using a strand-displacing DNA polymerase capable of carrying out isothermal amplification according to claim 9, wherein the amplification reaction is a rolling circle amplification (RCA) reaction.

14. The method of amplifying a template DNA molecule using a strand-displacing DNA polymerase capable of carrying out isothermal amplification according to claim 9, wherein the template DNA is a circular DNA molecule.

15. The method of amplifying a template DNA molecule using a strand-displacing DNA polymerase capable of carrying out isothermal amplification according to claim 9, wherein the extension step is carried out in an isothermal temperature range with a temperature variation of 10° C. or less.

16. The method of amplifying a template DNA molecule using a strand-displacing DNA polymerase capable of carrying out isothermal amplification according to claim 9, wherein the extension step is carried out in a temperature range of 60° C. or less.

17. The method of amplifying a template DNA molecule using a strand-displacing DNA polymerase capable of carrying out isothermal amplification according to claim 9, wherein the extension step is carried out in a temperature range of 25 to 42° C.
